(19) United States
(12) Patent Application Publication
(10) Pub. No.: US 2006/0281981 A1
(43) Pub. Date: Dec. 14, 2006

(54) Apparatus, method, and medium of measuring skin hydration using mobile terminal

(75) Inventors: Woo-young Jang, Seoul (KR); Kyung-ho Kim, Yongin-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/433,354

(22) Filed: May 15, 2006

(30) Foreign Application Priority Data
Jun. 4, 2005 (KR) 10-2005-0048106

(51) Int. Cl. A61B 5/00

(52) U.S. Cl. 600/306

(57) ABSTRACT

An apparatus, method, and medium of measuring skin hydration using a mobile terminal such as a cell phone are provided. The apparatus for measuring skin hydration, which is included in a mobile terminal, includes: a voltage applying unit which receives a power from a power source included in the mobile terminal, and applies a single power source voltage to a measuring portion; a current measuring unit which measures a current flowing through the measuring portion where the voltage is applied; a voltage amplifier which is input the measured current through a resistor, and amplifies a voltage across the resistor; a control unit which controls an output voltage of the voltage amplifier to be in predetermined range; and a calculating unit which calculates a susceptance of the measuring portion using the output voltage of the voltage amplifier, and calculates the skin hydration of the measuring portion using the calculated susceptance. Accordingly, skin hydration is measured using a single power source having a narrow voltage range used in a mobile terminal, so that a user can measure his or her skin condition at anytime and anyplace.

### CROSS-REFERENCE TO RELATED APPLICATIONS

This application claims the benefit of Korean Patent Application No.10-2005-0048106, filed on Jun. 4, 2005, in the Korean Intellectual Property Office, the disclosure of which is incorporated herein in its entirety by reference.

### BACKGROUND OF THE INVENTION

1. Field of the Invention

The present invention relates to an apparatus, method, and medium of measuring skin hydration, and more particularly, to an apparatus, method, and medium of measuring skin hydration using a mobile terminal such as a cell phone or personal digital assistant.

2. Description of the Related Art

Skin contains moisture, which functions as a skin barrier for protecting a human body from an external stimulus and infection. Recently, with the increase of interest in skin care, apparatuses for measuring skin hydration, that is, the amount of moisture included in a stratum corneum of skin, are being developing. The skin hydration measured by the apparatuses may be used in choosing toiletries, estimating influence of temperature and humidity in surrounding environment, and diagnosing dermatological diseases.

FIG. 1 is a cross-sectional view of a skin structure. Skin is composed of a stratum disjunctum 100, a stratum corneum 110, a stratum spinosum 120, a dermis 130, and a subcutaneous tissue 140. An epidermis includes the stratum disjunctum 100, the stratum corneum 110, and the stratum spinosum 120, and prevents the skin hydration from evaporation so that the skin hydration is balanced and the skin barrier function can be maintained. The hydration of the epidermis is balanced by the process of maintaining the hydration of the stratum corneum 110.

The skin hydration means the amount of moisture included in the stratum corneum 110, and is an optimal standard for indicating a condition of the skin barrier. The skin hydration varies depending on individuals, body parts, and seasons.

In a method of measuring the skin hydration, a voltage is applied to the skin, and a current flowing through the skin according to the voltage is measured to calculate the hydration of the stratum corneum 110.

However, a conventional measuring apparatus (e.g., conventional measuring device) using the above method is powered by a regular power supply providing 220V or 110V. The conventional measuring apparatus converts 220V or 110V to 12V and supplies 12V to a measuring portion. However, since apparatuses using the above method are not easily portable, a user cannot measure his or her skin condition at anytime and anyplace. In addition, to measure the skin hydration by using a mobile terminal such as a cell phone with the measuring portion requiring 12V, a power source of about 3V included in the mobile terminal has to be boosted up using a DC/DC converter to boost 3V to 12V, resulting in high power consumption.

### SUMMARY OF THE INVENTION

Additional aspects, features, and/or advantages of the invention will be set forth in part in the description which follows and, in part, will be apparent from the description, or may be learned by practice of the invention.

The present invention provides an apparatus, method, and medium of measuring skin hydration using a single power source included in a mobile terminal. For example, the single power source may be about 3 volts.

According to an aspect of the present invention, there is provided an apparatus for measuring skin hydration, which is included in a portable terminal, comprising: a voltage applying unit which receives a power from a power source included in the portable terminal, and applies a single power source voltage to a measuring portion; a current measuring unit which measures a current flowing through the measuring portion where the voltage is applied; a voltage amplifier which receives the measured current through a resistor, and amplifies a voltage across the resistor; a control unit which controls an output voltage of the voltage amplifier to be in a predetermined range; and a calculating unit which calculates a susceptance of the measuring portion using the output voltage of the voltage amplifier, and calculates the skin hydration of the measuring portion using the calculated susceptance.

In the aforementioned aspect of the present invention, the predetermined range may be a measurable voltage range of the portable terminal.

The control unit may control an amplifying ratio of the voltage amplifier, so that the output voltage of the voltage amplifier belongs to the predetermined range.

The control unit may control the single power source voltage of the voltage applying unit, so that the output voltage of the voltage amplifier belongs to the predetermined range.

The voltage applying unit may further comprises an amplifier which amplifies the single power source voltage at an amplifying ratio of greater than 0 and less than 1, wherein the control unit controls the amplifying ratio of the amplifier.

The control unit may divide an output voltage of the voltage amplifier into two or more sections, and applies different amplifying ratios to the sections.

The voltage amplifier may further comprise two or more amplifiers.

The voltage amplifier may be a non-linear amplifier.

The voltage applying unit may apply a single power source voltage, which is different from the voltage applied to the measuring portion, to a capacitor or an inductor, when the apparatus for measuring skin hydration is calibrated.

According to another aspect of the present invention, there is provided a method of measuring skin hydration using a mobile terminal, comprising: receiving a power from a power source included in the mobile terminal, and applying a single power source voltage to a measuring portion; measuring a current flowing through the measuring portion where the voltage is applied; amplifying a voltage across a resistor through which the measured current flows; calculating a susceptance of the measuring portion using the amplified output voltage; and calculating the skin hydration of the measuring portion using the calculated susceptance.

The predetermined range may be a measurable voltage range of the portable terminal.

Before the amplifying a voltage, the method may further comprise controlling an amplifying ratio so that the amplified voltage is in a predetermined range

The applying a single power source voltage amplified voltage may further comprise controlling the single power source voltage so that the amplified voltage is in a predetermined range

Before the amplifying a voltage, the method may further comprise dividing an amplified voltage into two or more sections and applying and different amplifying ratio to the sections so that the amplified voltage after the amplify a voltage is in a predetermined range

In the amplifying a voltage, the voltage may be divided into two or more steps before being amplified.

In the amplifying a voltage, the voltage may be amplified using a non-linear amplifier.

The present invention also provides a computer-readable medium having embodied thereon a computer program for executing the method above.

According to another aspect of the present invention, there is provided a mobile terminal for measuring hydration of a measuring portion of skin, comprising: a voltage applying unit which applies an AC voltage to the measuring portion of the skin to be measured; a current measuring unit which measures a current flowing through the measuring portion of the skin; a voltage amplifier which receives the measured current through a resistor, and amplifies a voltage corresponding to the measured current; a control unit which controls the amplified voltage so that the amplified voltage does not exceed a maximum measurable voltage; and a calculation unit which calculates a susceptance of the measuring portion using the amplified voltage, and calculating the skin hydration of the measuring portion using the calculated susceptance.

The mobile terminal may be a cell phone.

The mobile terminal may be a personal digital assistant.

According to another aspect of the present invention, there is provided a method of measuring skin hydration of a measuring portion of skin using a mobile terminal, comprising: measuring a current flowing through a measuring portion where the voltage is applied; calculating a susceptance of the measuring portion based on the measured current; and calculating the skin hydration of the measuring portion using the calculated susceptance.

The method may further comprise applying a single power source voltage from the mobile terminal to a measuring; portion of skin, which is to be measured to determine its skin hydration; and amplifying a voltage across a resistor through which the measured current flows, wherein the susceptance is calculated based on the amplified voltage, which is based on the measured current.

The mobile terminal may be a cell phone.

The mobile terminal may be a personal digital assistant.

According to another aspect of the present invention, there is provided at least one computer readable medium storing instructions that control at least one processor to perform a method comprising: measuring a current flowing through a measuring portion where the voltage is applied; calculating a susceptance of the measuring portion based on the measured current; and calculating the skin hydration of the measuring portion using the calculated susceptance.

The mobile terminal may be a cell phone.

The mobile terminal is a personal digital assistant.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a cross-sectional view of a skin structure;

FIG. 2 shows a method of measuring skin hydration, applied to an apparatus for measuring skin hydration, according to an exemplary embodiment of the present invention;

FIG. 3 is a block diagram of a structure of a mobile terminal capable of measuring skin hydration, according to an exemplary embodiment of the present invention;

FIG. 4 is a graph illustrating the operation of a non-linear amplifier; and

FIG. 5 is a flowchart illustrating a method of measuring skin hydration using a mobile terminal, according to an exemplary embodiment of the present invention.

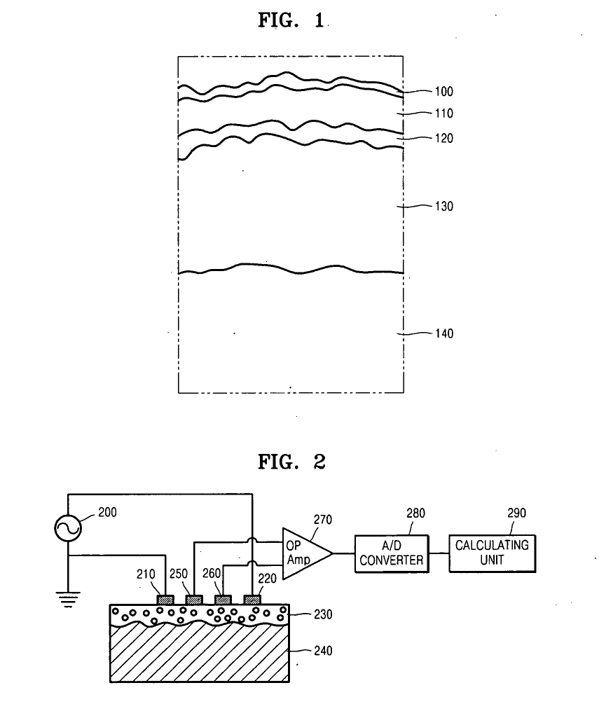

### DETAILED DESCRIPTION OF THE PREFERRED EMBODIMENTS

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

FIG. 2 shows a method of measuring skin hydration, applied to an apparatus for measuring skin hydration, according to an exemplary embodiment of the present invention. An AC power source 200 apply a voltage to the skin composed of an epidermis 230 and a dermis 240 through two voltage applying electrodes 210 and 220 attached to skin. The voltage may have a low frequency about 50 KHz in order to measure a susceptance of a stratum corneum included in the epidermis 230.

A current flowing through the skin according to the applied voltage is measured through two measuring electrodes 250 and 260, and a voltage corresponding to the current flowing through the skin is amplified by an op-Amp 270 to be output. The amplified voltage is converted to a digital signal through an A/D converter 280. A calculating unit 290 calculates the susceptance of the stratum corneum using the converted digital signal. The calculation unit 290 calculates the hydration of the stratum corneum using the calculated susceptance.

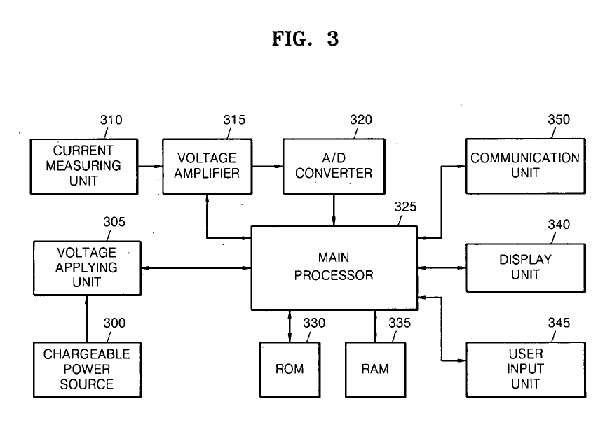

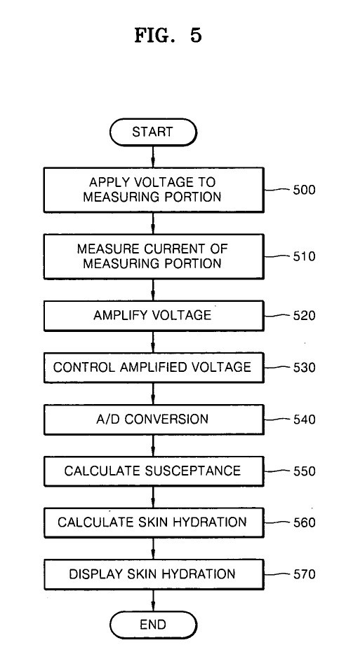

FIG. 3 is a block diagram of a structure of a mobile terminal capable of measuring skin hydration, according to an exemplary embodiment of the present invention. Examples of a mobile terminal include a cell phone or personal digital assistant. The mobile terminal includes a chargeable power source 300, a voltage applying unit 305, a current measuring unit 310, a voltage amplifier 315, an A/D converter 320, a main processor 325, a ROM 330, a RAM 335, a display unit 340, a user input unit 345, and a communication unit 350. The operation of the apparatus for measuring skin hydration using the mobile terminal of FIG. 3 will now be described with reference to FIG. 5.

The communication unit 350 transmits and receives data or a voice signal to/from a base station, under the control of the main processor 325. The display unit 340 displays a condition of the mobile terminal. The user input unit 345 receives inputs for performing specific functions related to the mobile terminal. The ROM 330 stores a program for operating the main processor 325.

The voltage applying unit 305 is provided a power from the chargeable power source 300, and applies a voltage to a measuring portion on skin whose hydration is to be measured (operation 500). The voltage applying unit 305 may include two op-Amps, and may apply a sine wave of voltage amplified by the op-Amps to the measuring portion. In general, a single power source in the range of 0˜3V is used in the mobile terminal. Thus, the voltage applying unit 305 also applies a single power source in the range of 0˜3V to the measuring portion. The voltage applying unit 305 may receive the sine wave of voltage used in the main processor 325 directly from the main processor 325, to apply the sine wave of voltage to the measuring portion. Although the single power source is generally in the range of 0˜3V and the voltage applying unit 305 can apply a voltage in this range, the single power source could be above 3 volts and the voltage applying unit 305 can apply a voltage above 3 volts.

The current measuring unit measures a current flowing through the measuring portion according to the applied voltage, through electrodes (not shown) attached to the measuring portion (operation 510). The measured current flows through a resistor (not shown) to form a voltage to be input to the voltage amplifier 315. Here, the resistor may have a resistance so that the amplified voltage output from the voltage amplifier 315 does not exceed the voltage range used in the mobile terminal. The voltage amplifier 315 amplifies a voltage corresponding to the measured current, using an amplifier such as an op-Amp (operation 520). When an amplitude of the amplified voltage exceeds a maximum of a measurable range, for example 0˜3V adopted by the mobile terminal, the amplitude of the amplified voltage becomes saturated to the maximum of the measurable range, for example 3V. Accordingly, the main processor 325 controls the output voltage of the voltage amplifier 315 to be in the measurable voltage range by the mobile terminal.

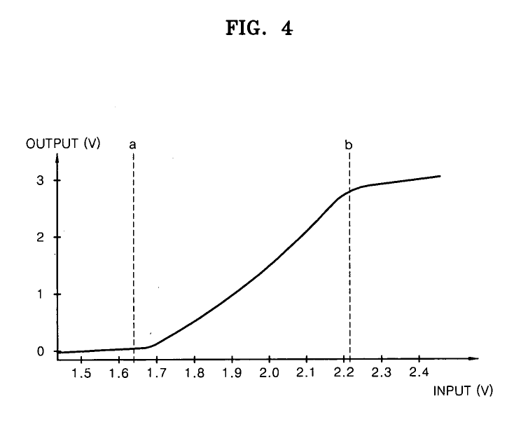

The voltage amplifier 315 may be a non-linear amplifier. A voltage range of the output of the voltage amplifier 315, which can be used in measuring skin hydration, belongs to a linear section where an output voltage increases in proportion to an input voltage. FIG. 4 illustrates input/output voltages of the non-linear amplifier. A linear section (a-b) can extend as shown in FIG. 4 by amplifying the input voltage using the non-linear amplifier. If a narrow range voltage, as adopted in the present invention, is amplified by a linear amplifier, it results in a narrow linear section and accordingly a high probability of skin hydration measurement errors. Therefore, employing the non-linear amplifier results in extension of the linear section and reduction of the skin hydration measurement errors.

To allow the voltage applying unit 305 to apply a voltage to the measuring portion, and the current measuring unit 310 to measure a current flowing through the measuring portion accurately, electrodes included in the voltage applying unit 305 and the current measuring unit 310 have to contact the measuring portion for more than a predetermined time. It is therefore desired that the display unit 340 display a time for measuring in order to let the user know how long the electrodes of the voltage applying unit 305 and the current measuring unit 310 come in contact with the measuring portion.

Hereinafter, processes of controlling the amplified voltage in operation 530, performed by the main processor 325, will be described in detail.

In a first process, the output voltage of the voltage amplifier 315 is input to the main processor 325, and then the main processor 325 adjusts an amplifying ratio of the voltage amplifier 315 so that the output voltage belongs to the measurable voltage range of the mobile terminal. The main processor 325 may adjust the amplifying ratio of the voltage amplifier 315 by adjusting the resistance of a variable resister included in the voltage amplifier 315. The greater the output voltage of the voltage amplifier 315, the greater a signal to noise ratio (SNR). Thus, for accurate measurement of the skin hydration, the main processor 325 may control the amplified voltage to be a maximum measurable voltage in the mobile terminal.

In a second process, the output voltage of the voltage amplifier 315 is divided into two sections, and an amplifying ratio corresponding to each section is stored in the RAM 335. The main processor 325 reads from the RAM 335 an amplifying ratio corresponding to the amplified voltage that is input from the voltage amplifier 315, and adjusts the amplifying ratio of the voltage amplifier 315. Assuming, for example, that the amplifying ratio of 100 times is stored in the RAM 335 when the output voltage belongs to 0˜1.5 V, and the amplifying ratio of 10 times is stored in the RAM 335 when the output voltage belongs to 1.5˜3 V, the main processor 325 may adjust the amplifying ratio of the voltage amplifier 315 to be 100 times when the output voltage of the voltage amplifier 315 is 0.5 V.

In a third process, the voltage applying unit 305 includes an amplifier (not shown) for reducing an amplitude of a voltage, whose amplifying ratio is greater than 0 and less than 1. The main processor 325 adjusts then amplifying ratio of the amplifier (not shown) so that the output voltage of the voltage amplifier 315 does not exceed the measurable voltage range of the mobile terminal.

In a fourth process, the voltage amplifier 315 includes two or more op-Amps, amplifies a voltage input to the voltage amplifier 315 in two or more steps to control the output voltage of the voltage amplifier 315 not to exceed the measurable voltage range of the mobile terminal. When the main processor 325 adjusts the amplifying ratio to control the output voltage of the voltage amplifier 315, the adjusted amplifying ratio may be stored in the RAM 335.

The A/D converter 320 converts the output voltage of the voltage amplifier 315 into a digital signal (operation 540). The main processor 325 receives the digital signal from the A/D converter, and calculates a susceptance B of the stratum corneum of the measuring portion according to the following equation 1.

$$\frac{I}{V} = Y = G - jB \qquad [ Equation 1 ]$$

Here, I is a measured current, V is an applied voltage, Y is an admittance of the measuring portion, G is a conductance of the measuring portion, and B is a susceptance of the measuring portion, where j is a notation used to identify imaginary number values.

Using the amplifying ratio of the voltage amplifier 315 and the magnitude of the digital signal input from the A/D converter 320, the main processor 325 calculates a current that is measured by the current measuring unit 310 using the resistance adopted before the voltage amplifier 315. The current is then divided by the voltage applied by the voltage applying unit 305, thereby calculating the susceptance of the measuring portion. Here, the frequency of the applied voltage is of a low frequency of about 50 KHz.

The main processor 325 calculates the skin hydration of the measuring portion by using the calculated susceptance of the measuring portion (operation 560). The relationship between the susceptance and the skin hydration is stored in the RAM 335 in the form of a relational equation or a look-up table. The main processor 325 may calculate the skin hydration of the measuring portion from the calculated susceptance by using the relationship between the susceptance and the skin hydration stored in the RAM 335. In addition, the relationship between the susceptance and the skin hydration may also be obtained by experiment.

The skin hydration calculated from the main processor 325 is input to the display unit 340, and is displayed through the display unit 340 (operation 570).

The ROM 330 may store information on skin to be provided to the user according to the measured skin hydration, for example, information on toiletries designed for specific skin types or information on skin conditions under an environment may be stored in the ROM 330. The main processor 325 may read the information on the calculated skin hydration from the ROM 330, to provide the information to the user through the display unit 345.

A calibration method for adjusting a degree of accuracy of a skin hydration measuring apparatus using the mobile terminal will now be described. In the method, to carry out calibration, the skin hydration measuring apparatus measures a susceptance using a capacitor or an inductor, where the capacitance and the inductance thereof are already known, and then it is checked whether the measured susceptance is equal to the capacitance or the inductance.

If a voltage, which is equal to the voltage applied to the measuring portion in measuring the skin hydration, is applied to the capacitor or the inductor to carry out calibration, in many cases, the output voltage of the voltage amplifier 315 exceeds the measurable voltage range of the mobile terminal. Thus, when performing calibration, a voltage less than the voltage applied in measuring the skin hydration may be applied by the voltage applying unit 305. Specifically, in two op-Amps used by the voltage applying unit 305 when generating an applying voltage, a sine wave of voltage is output from one op-Amp to measure the skin hydration, and a cosine wave of voltage is output from the other op-Amp. Here, the cosine wave of voltage may be applied to the capacitor or the inductor.

Accordingly, in an apparatus and method of measuring skin hydration using a mobile terminal, skin hydration is measured using a single power source used in a mobile terminal, so that a user can measure his or her skin condition at anytime and anyplace. The single power source can be about 3V.

In addition to the above-described exemplary embodiments, exemplary embodiments of the present invention can also be implemented by executing computer readable code/instructions in/on a medium, e.g., a computer readable medium. The medium can correspond to any medium/media permitting the storing and/or transmission of the computer readable code.

The computer readable code/instructions can be recorded/transferred in/on a medium in a variety of ways, with examples of the medium including magnetic storage media (e.g., ROM, floppy disks, hard disks, etc.), optical recording media (e.g., CD-ROMs, or DVDs), random access memory media, and storage/transmission media such as carrier waves. Examples of storage/transmission media may include wired or wireless transmission (such as transmission through the Internet). The medium may also be a distributed network, so that the computer readable code/instructions is stored/transferred and executed in a distributed fashion. The computer readable code/instructions may be executed by one or more processors.

Although a few exemplary embodiments of the present invention have been shown and described, it would be appreciated by those skilled in the art that changes may be made in these exemplary embodiments without departing from the principles and spirit of the invention, the scope of which is defined in the claims and their equivalents.

What is claimed is:

1. An apparatus for measuring skin hydration, which is included in a mobile terminal, comprising:
a voltage applying unit which receives a power from a power source included in the mobile terminal, and applies a single power source voltage to a measuring portion;
a current measuring unit which measures a current flowing through the measuring portion where the voltage is applied;
a voltage amplifier which receives the measured current through a resistor, and amplifies a voltage across the resistor;
a control unit which controls an output voltage of the voltage amplifier to be in a predetermined range; and
a calculating unit which calculates a susceptance of the measuring portion using the output voltage of the voltage amplifier, and calculates the skin hydration of the measuring portion using the calculated susceptance.

2. The apparatus of claim 1, wherein the predetermined range is a measurable voltage range of the mobile terminal.

3. The apparatus of claim 1, wherein the control unit controls an amplifying ratio of the voltage amplifier, so that the output voltage of the voltage amplifier belongs to the predetermined range.

4. The apparatus of claim 1, wherein the control unit controls the single power source voltage of the voltage applying unit, so that the output voltage of the voltage amplifier belongs to the predetermined range.

5. The apparatus of claim 1, the voltage applying unit further comprising an amplifier which amplifies the single power source voltage at an amplifying ratio of greater than 0 and less than 1,
wherein the control unit controls the amplifying ratio of the amplifier.

6. The apparatus of claim 3, wherein the control unit divides an output voltage of the voltage amplifier into two or more sections, and applies different amplifying ratios to the sections.

7. The apparatus of claim 1, wherein the voltage amplifier further comprises two or more amplifiers.

8. The apparatus of claim 1, wherein the voltage amplifier is a non-linear amplifier.

9. The apparatus of claim 1, wherein the voltage applying unit applies a single power source voltage, which is different from the voltage applied to the measuring portion, to a capacitor or an inductor, when the apparatus for measuring skin hydration is calibrated.

10. A method of measuring skin hydration using a mobile terminal, comprising:
receiving a power from a power source included in the mobile terminal, and applying a single power source voltage to a measuring portion;
measuring a current flowing through the measuring portion where the voltage is applied;
amplifying a voltage across a resistor through which the measured current flows;
calculating a susceptance of the measuring portion using the amplified output voltage; and
calculating the skin hydration of the measuring portion using the calculated susceptance.

11. The method of claim 10, wherein the predetermined range is a measurable voltage range of the mobile terminal.

12. The method of claim 10, wherein, before the amplifying a voltage, further comprises controlling an amplifying ratio so that the amplified voltage is in a predetermined range

13. The method of claim 12, wherein the applying a single power source voltage further comprises controlling the single power source voltage so that the amplified voltage is in a predetermined range.

14. The method of claim 12, wherein, before the amplifying a voltage, further comprises dividing an amplified voltage into two or more sections, and applying and different amplifying ratios to the sections.

15. The method of claim 10, wherein, in the amplifying a voltage, the voltage is divided into two or more steps before being amplified.

16. The method of claim 10, wherein, in the amplifying a voltage, the voltage is amplified using a non-linear amplifier.

17. A computer-readable medium having embodied thereon a computer program for executing the method of claim 10.

18. A mobile terminal for measuring hydration of a measuring portion of skin, comprising:
a voltage applying unit which applies an AC voltage to the measuring portion of the skin to be measured;
a current measuring unit which measures a current flowing through the measuring portion of the skin;
a voltage amplifier which receives the measured current through a resistor, and amplifies a voltage corresponding to the measured current;
a control unit which controls the amplified voltage so that the amplified voltage does not exceed a maximum measurable voltage; and
a calculation unit which calculates a susceptance of the measuring portion using the amplified voltage, and calculating the skin hydration of the measuring portion using the calculated susceptance.

19. The mobile terminal of claim 18, wherein the mobile terminal is a cell phone.

20. The mobile terminal of claim 18, wherein the mobile terminal is a personal digital assistant.

21. A method of measuring skin hydration of a measuring portion of skin using a mobile terminal, comprising:
measuring a current flowing through a measuring portion where the voltage is applied;
calculating a susceptance of the measuring portion based on the measured current; and
calculating the skin hydration of the measuring portion using the calculated susceptance.

22. The method of claim 21, further comprising:
applying a single power source voltage from the mobile terminal to a measuring portion of skin, which is to be measured to determine its skin hydration; and
amplifying a voltage across a resistor through which the measured current flows,
wherein the susceptance is calculated based on the amplified voltage, which is based on the measured current.

23. The method of claim 21, wherein the mobile terminal is a cell phone.

24. The method of claim 21, wherein the mobile terminal is a personal digital assistant.

25. At least one computer readable medium storing instructions that control at least one processor to perform a method comprising:
measuring a current flowing through a measuring portion where the voltage is applied;
calculating a susceptance of the measuring portion based on the measured current; and
calculating the skin hydration of the measuring portion using the calculated susceptance.

26. At least one computer readable medium as recited in claim 25, wherein the mobile terminal is a cell phone.

27. At least one computer readable medium as recited in claim 25, wherein the mobile terminal is a personal digital assistant.
